(19) United States
(12) Patent Application Publication
(10) Pub. No.: US 2016/0157772 A1
(43) Pub. Date: Jun. 9, 2016

(54) Neuropathic Assistive Device

(71) Applicant: Duncan M. Grant, Hebron, OH (US)

(72) Inventor: Duncan M. Grant, Hebron, OH (US)

(21) Appl. No.: 14/944,848

(22) Filed: Nov. 18, 2015

Related U.S. Application Data
(63) Continuation of application No. 14/851,340, filed on Sep. 11, 2015.
(60) Provisional application No. 62/048,998, filed on Sep. 11, 2014.

(51) Int. Cl. A61B 5/00; A61B 5/145; A61B 90/98; A61B 5/01; A61B 5/103; A61B 5/1468; A61B 5/0492

(52) U.S. Cl. CPC A61B 5/486; A61B 5/1468; A61B 5/14539; A61B 5/0059; A61B 5/0492; A61B 5/01; A61B 5/746; A61B 5/1032; A61B 5/742; A61B 5/7455; A61B 5/7475; A61B 5/7405; A61B 5/0002; A61B 5/6898; A61B 5/6897; A61B 90/98; A61B 2090/064

(57) ABSTRACT

Methods, systems, and apparatuses are disclosed for neuropathic assistance. In one embodiment, a neuropathic assistance system senses and detects at least one of: an inflammation condition, a wound, and a wound-causing environmental factor in an affected tissue, that may cause at least one of: a visual alert, a tactile alert, and an audio alert, to be output in response to a sensing and detection of at least one of: an inflammation condition, a wound, and a wound-causing environmental factor.

This application claims priority as a continuation of U.S. Nonprovisional patent application Ser. No. 14/851,340, filed on Sep. 11, 2015, which claims priority from U.S. Provisional Patent Application No. 62/048,998, filed on Sep. 11, 2014, each of which is incorporated by reference herein in its entirety.

### BACKGROUND

Diabetes mellitus, commonly referred to as simply “diabetes” is a metabolic disease that affects millions worldwide and is a leading cause of death.

Management and treatment of diabetic symptoms may be necessary to prevent long-term complications caused by diabetes. Serious long-term complications of diabetes may include kidney damage, blindness and other eye damage, heart disease, stroke, foot ulcers, and limb amputations.

Long-term diabetic complications may arise from damage to blood vessels. Damage to blood vessels may affect blood flow, which in turn may affect other body systems such as the nervous system. Neuropathy, for example diabetic neuropathy, a common complication of diabetes, may cause damage to nerves. Neuropathy symptoms and complications may include pain, numbness, and tingling in different areas of the body, which may in turn lead to other complications such as damage to skin and other tissue.

Healthy tissue may be damaged by diseases and conditions with vascular and neuropathic complications, for example, like those of diabetes. Scratches, blisters, cuts, and the like, collectively referred to herein as “inflammation conditions,” are common occurrences on people at every level of health. Environmental factors such as chaffing, chapping, exposure to irritants, allergic reactions, sunburn, pressure on tissue, friction on tissue, and the like, may cause inflammation conditions on skin tissue of people at all levels of health. If unnoticed, or left untreated and unmonitored, inflammation conditions may exacerbate into sores, lesions, ulcers, and the like, referred to herein collectively as “wounds.” Inflammation conditions in patients with certain medical conditions and complications, for example, conditions and complications experienced by diabetic patients, may develop into wounds that if unmonitored and untreated, may cause more serious long-term complications and health risks. A patient with a neuropathic condition, for example a diabetic patient with symptoms of diabetic neuropathy, may not feel a development and formation of a wound on a remote area of the body, such as on a foot or on an area of the back, and may not be able to visibly and tactilely detect inflammation conditions, wounds, and any underlying environmental factors that may cause inflammation conditions and wounds. Certain diseases and conditions, for example, diabetes, may include other complications such as diabetic vascular complications, that may affect a healing ability and healing speed of an inflammation condition and a wound. A patient with a neuropathic condition may be unable to detect inflammation conditions and wound formation on certain areas of the body, such that any inflammation condition and wound, if left undetected and untreated, may cause damage to a tissue, and may cause a damaged tissue to be amputated to treat the affected tissue.

Currently, prevention and treatment of wounds in patients with neuropathic conditions may include at least one of: regular doctor visits; proper hygiene to treat wounds and inflammation conditions; proper hygiene and behavior to limit environmental factors that may cause wounds and inflammation conditions; and specialized clothing, for example, special diabetic footwear including socks and shoes. Current prevention and treatment methods and devices used to prevent and treat wounds in patients with neuropathic and like conditions may have limitations.

The present application is directed to novel systems and methods for neuropathic assistance that may detect and alert to a presence of at least one of: inflammation conditions, wounds, and wound-causing environmental factors that may cause and exacerbate inflammation conditions and wounds.

### SUMMARY

Systems and methods for neuropathic assistance that may detect and alert to a presence of at least one of: an inflammation condition, wounds, and wound-causing environmental factors, are provided.

In one embodiment, a neuropathic assistance system is provided, the neuropathic assistance system comprising: a sensor in operable contact with an affected tissue and operable to sense and detect a presence of at least one of: an inflammation condition, a wound, and a wound-causing environmental factor, at a location on or near the affected tissue; a control system; and an alert device.

In another embodiment, a neuropathic assistance system is provided, the neuropathic assistance system comprising: a local neuropathic assistance system in operable contact with a user, the local neuropathic assistance system further comprising: a sensor in operable contact with an affected tissue and operable to sense and detect at least one of: an inflammation condition, a wound, and a wound-causing environmental factor, and provide a sensor signal; a first control system, an alert device, and a first communication hardware; a remote neuropathic assistance system; the remote neuropathic assistance system operable to wirelessly receive the sensor signal, the remote neuropathic assistance system further comprising: a second control system, a second communication hardware, and at least one of: an electronic display, a vibration motor, and a speaker, wherein the first communication hardware is operable to wirelessly communicate with the second communication hardware.

In another embodiment, a method for providing neuropathic assistance is provided, the method comprising: providing a sensor in operable contact with an affected tissue to sense and detect a presence of at least one of: an inflammation condition, a wound, and a wound-causing environmental factor; sensing and detecting at least one of: the presence, and an absence, of at least one of: the inflammation condition, the wound, and the wound-causing environmental factor, to produce a sensor signal; transferring the sensor signal to a processing device for comparison against a predefined threshold value associated with the at least one of: the inflammation condition, the wound, and the wound-causing environmental factor; comparing the sensor signal against the predefined threshold value to determine at least one of: an excess value, and a normal value; generating an actuator signal in response to an excess value determination; and transferring the actuator signal to at least one of: a display, a visual alert out, a vibration motor, a tactile alert output, a speak, and an audio alert output, to alert a user to the presence of at least one of: an inflammation condition, a wound, and a wound-causing environmental factor, using at least one of: a visual alert, a tactile alert, and an audio alert.

### BRIEF DESCRIPTION OF THE DRAWINGS

The accompanying figures, which are incorporated in and constitute a part of the specification, illustrate various example systems and methods, and are used merely to illustrate various example embodiments.

FIG. 1A illustrates an example inflammation condition.

FIG. 1B illustrates an example wound development.

FIG. 2 illustrates a schematic view of an example neuropathic assistance system.

FIG. 3 illustrates a perspective view of an example neuropathic assistance system.

FIG. 4 illustrates a perspective view of an example neuropathic assistance system.

FIG. 5 illustrates a schematic view of an example neuropathic assistance system.

FIG. 6 illustrates a perspective view of an example neuropathic assistance system.

FIG. 7 illustrates a perspective view of an example neuropathic assistance system.

FIG. 8 illustrates a schematic view of an example neuropathic assistance system.

FIG. 9 is a flow chart of an example method for neuropathic assistance.

### DETAILED DESCRIPTION

Embodiments claimed herein disclose example neuropathic assistive systems, apparatuses, and methods to at least one of: limit and prevent, damage to tissue systems affected by neuropathy. As illustrated and described herein, neuropathic assistive systems, apparatuses, and methods may detect and alert a user and others to a presence of at least one of: an inflammation condition, a wound, and a wound-causing environmental factor, all of which may affect and damage tissue systems affected by neuropathy, for example, skin tissue on a diabetic foot. While embodiments illustrated and described herein may describe certain tissues on certain areas of a user, example embodiments are in no way limiting to the scope of any neuropathic assistive systems, apparatuses, and methods, as all may be adapted to detect and alert a presence of at least one of: an inflammation condition, a wound, and a wound-causing environmental factor, in other tissues, on other locations on a user. While certain diseases and conditions may be described with neuropathic complications, for example, diabetes, neuropathic assistive systems, apparatuses, and methods, as described herein, may be used to detect and alert a presence of at least one of: an inflammation condition, a wound, and a wound-causing environmental factor for other diseases and conditions that may cause inflammation conditions and wounds, such as arteriosclerosis. Likewise, neuropathic assistive systems, apparatuses, and methods, as described herein, may be used to detect and alert a presence of at least one of: inflammation conditions, wounds, and wound-causing environmental factors, in other non-neuropathic diseases and conditions that may limit detection of inflammation conditions, wounds, and wound-causing environmental factors, such as blindness, paralysis, symptoms of advanced aging, and the like.

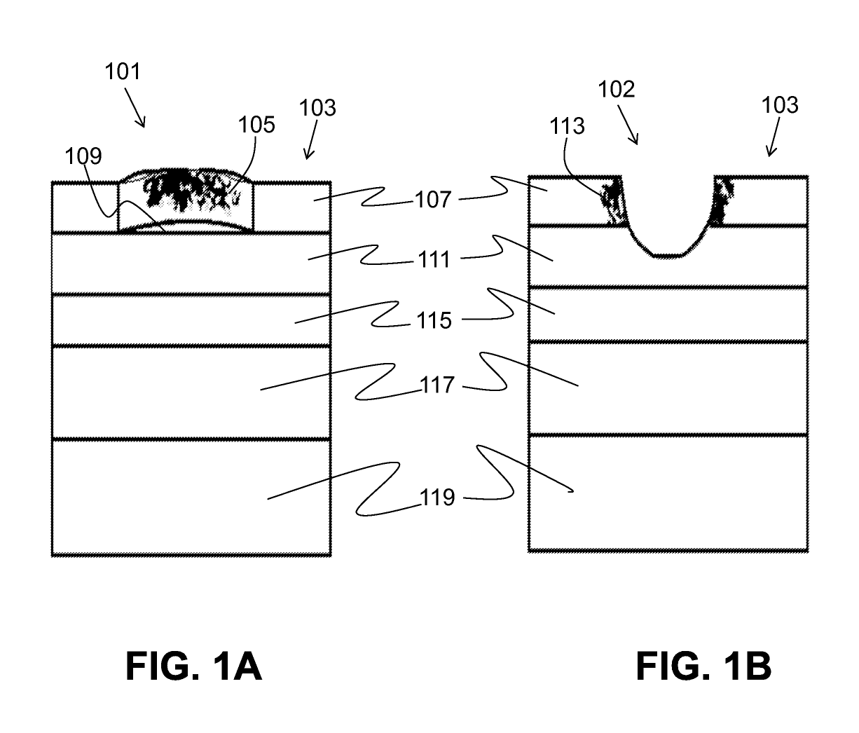

With reference to FIGS. 1A and 1B, example inflammation conditions 101 leading generally to wound 102, are illustrated. Wound 102 may be an ulcer, for example a pressure ulcer, decubitus ulcer, bedsore, venous and arterial ulcers, diabetic ulcers, lesions and the like, that may form on soft tissue 103 as a result of at least one of: pressure on soft tissue 103, shear forces (friction) on soft tissue 103, and other factors affecting blood flow to soft tissue 103. Wound 102 may form on soft tissue 103 such as skin tissue 103. Wound 102 may form on soft tissue 103 over a bony prominence, for example, on soft tissue 103 on at least one of: a back area, a heel area, a hip area, an elbow area, a knee area, an ankle area, and a head area. Pressure and friction on soft tissue 103 may affect blood flow to soft tissue 103. With reference to FIG. 1A, a cutaway of tissue layers for skin, fat, muscle, and bone tissues is illustrated. Pressure and friction on epidermis 107 of skin tissue 103 may affect blood flow to skin tissue 103, and may cause ischemia, hypoxia, edema, inflammation 105, and eventually necrosis 113, in skin tissue 103. Restricted blood flow to skin tissue 103 may cause inflammation 105 to form on, and in epidermis 107, and blister 109 to form between epidermis 107 and dermis 111. With reference to FIG. 1B, continued exposure to wound-causing environmental factors may continue to affect blood flow to skin tissue 103, and further exacerbate inflammation 105 and blister 109, in and on skin tissue 103, that may cause loss of dermis 111, and necrosis 113 in epidermis 107, as wound 102 begins to form in epidermis 107 and dermis 111. Continued exposure to wound-causing environmental factors that may affect blood flow to skin tissue 103 may cause wound 102 to progress through subcutaneous tissue (fat) 115, muscle 117, and into bone 119. Early detection of inflammation conditions and wound 102 may be necessary to provide adequate treatment to inflammation conditions and wound 102, and to allow adequate time for soft tissue 103 to heal. Neuropathy may limit and inhibit nociception—a body's ability so sense potential wound-causing environmental factors, and may also limit and inhibit pain perception that may make detection of wound 102 difficult. As neuropathic conditions may affect a body's tactile perception and somatic senses, a person with neuropathic conditions may have cause to use a neuropathic assistive system, and practice neuropathic assistive methods, that may help to detect and alert a neuropathic user or another to at least one of: an inflammation condition, a wound, and a wound-causing environmental factor.

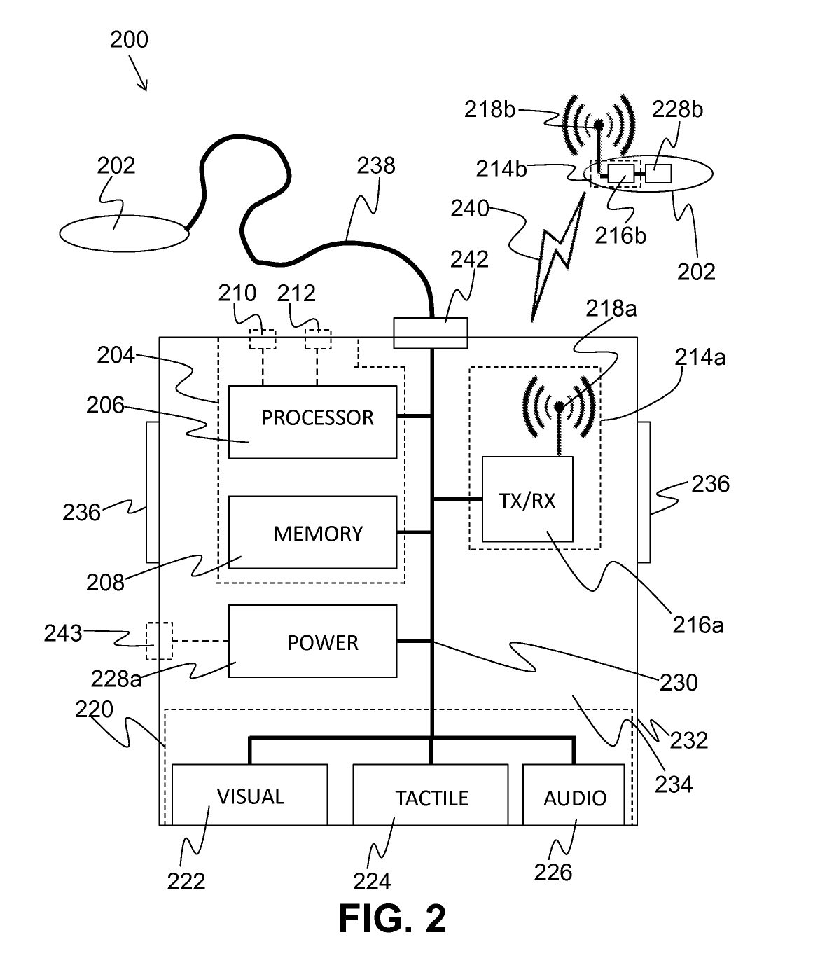

With reference to FIG. 2, an example schematic of a neuropathic assistive system 200 is illustrated. Neuropathic assistive system 200 may comprise: a sensor 202; a control system 204 that may further comprise processor 206, memory 208, input 210, and output 212; a wireless communication hardware 214a that may further comprise processor transmit/receive device 216a, and antenna 218a; an alert device 220 that may further comprise at least one of: a visual alert output 222, a tactile alert output 224, and an audio alert output 226; a power source 228a; a wired connection jack 242, and a power connection 243. As used herein, sensor 202, control system 204, processor 206, memory 208, input 210, output 212, wireless communication hardware 214a, transmit/receive device 216a, antenna 218a, alert device 220, visual alert output 222, tactile alert output 224, audio alert output 226, and power source 228a, wired connection jack 242, and power connection 243 may all be referred to collectively as “components” of neuropathic assistive system 200. Neuropathic assistive system 200 may further comprise interconnection hardware 230 to interconnect any component with another component; a wired connection 238 that may wiredly connect sensor 202 to other components of neuropathic system 200; and a wireless connection 238 that may wirelessly connect sensor 202 to other components of neuropathic assistive system 200.

Neuropathic assistive system 200 may be used to sense and detect a presence of at least one of: an inflammation condition, a wound, and a wound-causing environmental factor, by a user with a neuropathic condition that may have an impaired ability to sense an inflammation condition, a wound, and a wound-causing environmental factor. In one embodiment, neuropathic assistive system 200 may be used to sense and detect a presence of at least one of: an inflammation condition, a wound, and a wound-causing environmental factor, on a tissue at a particular body location on a user suffering from diabetes. In this embodiment, neuropathic assistive system 200 may be used to sense and detect at least one of: an inflammation condition, a wound, and a wound-causing environmental factor, for example, on a diabetic foot. In this embodiment, swollen feet, blisters, and soft tissue inflammation, for example, may be inflammation conditions; lesions and ulcers, for example, may be wounds; and pressure, friction, temperature, and moisture, for example, may be wound-causing environmental factors. In another embodiment, neuropathic assistive system 200 may be used to sense and detect a presence of at least one of: an inflammation condition, a wound, and a wound-causing environmental factor, by a user with leprosy. In another embodiment, neuropathic assistive system 200 may be used to sense and detect a presence of at least one of: an inflammation condition, a wound, and a wound-causing environmental factor, by a user with arteriosclerosis. In another embodiment, neuropathic assistive system 200 may be used to sense and detect a presence of at least one of: an inflammation condition, a wound, and a wound-causing environmental factor, by a blind user or user with limited vision. In another embodiment, neuropathic assistive system 200 may be used to sense and detect a presence of at least one of: an inflammation condition, a wound, and a wound-causing environmental factor, by a user with limited sensory capabilities caused by advanced aging. In another embodiment, neuropathic assistive system 200 may be used to sense and detect a presence of at least one of: an inflammation condition, a wound, and a wound-causing environmental factor, by a user that may be at least one of: paralyzed, disabled, bedridden, wheelchair-bound, and incapacitated. In another embodiment, neuropathic assistive system 200 may be used to sense and detect a presence of at least one of: an inflammation condition, a wound, and a wound-causing environmental factor, by a user with limited somatic sensory abilities and tactile perception. Neuropathic assistive system 200 may not be limited to use in detecting a presence of at least one of: an inflammation condition, a wound, and a wound-causing environmental factor, at certain tissue locations on a user, and may not be limited to detection of certain conditions for specific diseases and conditions, but rather neuropathic assistive system 200 may be used on any tissue at any location on the body, and may be used for any disease or condition that may cause a user to have a limited ability to sense and detect at least one of: an inflammation condition, a wound, and a wound-causing environmental factor.

Sensor 202 may be operable to sense and detect a presence of at least one of: an inflammation condition, a wound, and a wound-causing environmental factor, and generate and output a sensor signal in response. Sensor 202 may be at least one of: wiredly connected to other components of neuropathic assistive system 200 via at least: wired connection 238, wired connection jack 242, and interconnection hardware 230; and wirelessly connected to other components of neuropathic assistive system via at least: wireless connection 240, wireless communication hardware 214a, and interconnection hardware 230. In one embodiment, sensor 202 may comprise: wireless communication hardware 214b further comprising transmit/receive device 216b, and antenna 218b; and power source 228b. Wireless communication hardware 214b may be used to wirelessly communicate a sensor signal to wireless communication hardware 214a. Power source 228b may be used to power sensor 202 so that sensor 202 may perform required sensing functions and operations, and may be used to power wireless communication hardware 214b, for example, to send signals to, and receive signals from, wireless communication hardware 214a.

By non-limiting example, sensor 202 may include necessary hardware to sense and detect at least one of: a force, a pressure, a contact, a friction, a flex, a moisture, a movement, a temperature, a proximity, an electrodermal activity, a chemical presence, a biological presence, a pH level, and a color change, any of which, may indicate a presence of at least one of: an inflammation condition, a wound, and a wound-causing environmental factor.

Neuropathic assistive system 200 may include a sensor 202, that may be capacitive, operate on a principle based on capacitive coupling, and may determine a sensor signal output based on measured changes in capacitance. Neuropathic assistive wound system 200 may include a sensor 202 that may be resistive, operate on a principle of electrical resistance, and may determine a sensor signal output based on changes in resistance. Neuropathic assistive wound system 200 may include a sensor 202 that may be capacitive in part, and may be resistive in part. In one embodiment, neuropathic assistive wound system 200 may include a sensor 202 that may use a principle other than capacitance and resistance to determine a sensor signal output. In one embodiment, sensor 202 may be a capacitive touch sensor such that contact with a tissue on a user may vary capacitance to provide an indication of at least one of: contact, position, and displacement of a tissue relative to sensor 202. In another embodiment, sensor 202 may be resistive sensor that may vary a voltage or current of a sensor signal output from sensor 202 relative to an amount of mechanical force sensed by sensor 202, for example, to measure pressure, contact, and flex.

Sensor 202 may be a force sensor that may sense a mechanical force such as pressure relative to an affected tissue. A pressure sensor 202 may be used to detect a pressure and force relative to a threshold value, for example, a threshold pressure that may affect blood flow to a tissue. A flex sensor 202 may be used to detect a flex. In one embodiment, flex sensor 202 may be applied to a tissue and may detect muscle contraction and muscle relaxation, for example, to detect if muscle contraction and muscle relaxation may cause an affected tissue to come in contact with a wound-causing environmental factor. In another embodiment, flex sensor 202 may be embedded in an item, for example a clothing item, whose contact with an affected tissue may be a wound-causing environmental factor, to determine movement of a clothing item relative to an affected tissue.

Sensor 202 may be a biomedical sensor 202 that may sense and detect a value related to at least one of: electromyography (EMG), a moisture, a fluid, a temperature, an electrodermal activity, a chemical presence, a biological presence, sound, vibration, and a pH level. In one embodiment, biomedical sensor 202 may be an EMG sensor that may sense muscle flex and relaxation that may sense if muscle movement may cause a tissue to contact a wound-causing environmental factor. In another embodiment, biomedical sensor 202 may be a moisture, a fluid, and an electrodermal sensor, that may be operable to sense a presence of at least one of: blood, urine, sweat, exudate such as pus, and the like, that may indicate an inflammation condition, a wound, or may indicate a wound-causing environmental factor, such as microclimate conditions (i.e. localized heat and moisture at an affected tissue) that may contribute to ulcer development. In another embodiment, a biomedical sensor 202 may be a temperature sensor that may sense changes in temperature relative to a tissue, or a surrounding environment that may provide an indication of wound and inflammation conditions: where an elevated affected tissue temperature may indicate a tissue's immune response to a wound and inflammation condition; and where a lower than normal tissue temperature may indicate dead tissue, for example, to detect growth and development of a wound. Temperature sensor 202 may also be used to indicate a wound-causing environmental factor such as: microclimate, that may contribute to ulcer development; and ambient temperature, that may be used to indicate temperature levels that may cause perspiration on or near affected tissue. Biological sensor 202 may be at least one of: a chemical sensor 202 and a pH sensor 202. A biological sensor 202 may be used, for example, to detect a presence of a bacteria, an enzyme, and the like, that may: indicate a presence of an inflammation condition and a wound; and detect a presence of bacteria that may be a wound-causing environmental factor, for example, to indicate poor hygiene on, or around an affected tissue. A chemical sensor 202 may be used to detect a presence of a chemical or compound 202 that may be used to indicate a presence of a wound, inflammation condition, or wound-causing environmental factor, for example, low oxygen levels in blood around a tissue. A pH level may be used to indicate a presence of a wound, inflammation condition, or wound-causing environmental factor, for example, by correlating a blood pH level to a wound-causing environmental factor. Biological sensor 202 may include at least one of: a sound sensor 202, and a vibration sensor 202, that may be used to detect blood movement (i.e. heartbeat) at capillary sites surrounding affected tissue.

Sensor 202 may be a Hall-effect sensor 202 that may detect a presence and change in magnetic field levels to provide an indication of a wound-causing environmental factor. For example, an item near, or in contact with an affected tissue may be embedded with a magnet such that an items location relative to a tissue may be determined, for example, to detect if an item may be contacting a tissue, applying pressure to a tissue, and causing friction on a tissue.

Sensor 202 may be a tribo-sensor 202 that may be used to detect a friction between an item on or near a tissue, and a tissue. A friction between an item, for example a clothing item near a tissue, may be a wound-causing environmental factor that may cause inflammation conditions, and wounds, to an affected tissue.

Sensor 202 may be an RFID sensor 202 that may detect a change in radio frequency relative to a tissue. For example, an RFID tag may be integrated in an item such as a clothing item near an affected tissue such that a proximity of a clothing item may be determined relative to an affected tissue, by sensing a signal strength of an RF wave relative to an affected tissue. Contact of a clothing item with a tissue may be a wound-causing environmental factor.

Sensor 202 may be a proximity sensor 202, such as an ultrasonic range finder 202, that may be able to determine a proximity of a wound-causing environmental factor, for example, a location and a displacement of an item that may cause a pressure or friction on a nearby tissue.

Sensor 202 may be a photodetector 202 that may be able to detect a color and a color change, for example, to determine: a presence of an inflammation condition (blanching of skin); a presence of a wound; and any changes to wound size which may indicate healing of a wound, and exacerbation of a wound.

Sensors 202 may be a chip or integrated circuit designed to be robust, lightweight, small, thin, with a low power consumption, that may be, for example, easily positioned on or near a tissue. Sensors 202 may be pre-programmed with a predetermined threshold value, that when exceeded, may cause sensor 202 to output a sensor signal. Sensor 202 may be operable to output a variable sensor signal output based on a degree level of what is sensed. For example, a resistive sensor 202 may vary a resistance based on an amount of mechanical force as measured by resistive sensor 202, to provide a sensor signal output of variable voltages and currents. An application of light pressure on resistive force sensors 202 may cause a sensor signal to be generated and output with a lower voltage, while application of heavy pressure to resistive force sensor 202 may cause an output signal with a higher voltage. A sensor signal output from sensor 202 may be one of, or both of, an analog sensor signal, and a digital sensor signal. Sensor 202 may be placed on or near an affected tissue based on: what is to be sensed, a user's needs, a medical practitioner's advice and opinion, and other design factors. In one embodiment, sensors 202 may be used to sense at least one of: inflammation conditions, wounds, and wound-causing environmental factors over all areas of a tissue, for example, all areas of skin on a foot. In another embodiment, sensor 202 may be placed in a customized manner, as may be determined by a user or a doctor/practitioner, for sensing and detecting at least on of: inflammation conditions, wounds, and wound-causing environmental factors, relative to a specific area of a tissue, for example, a heel of a foot. In this embodiment, a doctor/practitioner, and a user may customize neuropathic assistive system 200 by defining specific placement of sensors 202 to sense and detect at least one of: inflammation conditions, wounds, and wound-causing environmental factors, on a specific location on a specific tissue. In other words, neuropathic assistive system 200 may be customized for each user.

Control system 204 may further comprise processor 206, memory 208, input 210, and output 212. Control system 204 may be used to at least one of: execute an instruction set to initiate a sensor reading from sensor 102; control a transmission of a sensor signal, and a control signal between one or more wireless communication hardware, for example, wireless communication hardware 214a, 214b; compare a sensor signal with a predetermined threshold to determine at least one of: an excess value, and a normal value, wherein an excess value may suggest a presence of at least one of: an inflammation condition, a wound, and a wound-causing environmental factor; in response to an excess value, outputting an actuator signal to at least one of: an actuator, an alert device 220, a visual alert output 222, a tactile alert output 224, an audio alert output 226, an electronic display (not shown), a speaker (not shown), and a vibration motor (not shown), to output at least one of: a visual alert, a tactile alert, and an audio alert.

Control system 204 may comprise processor 206 and may be used for a variety of functions in neuropathic assistive system 200. Processor 206 may be chosen based on intended function and design of neuropathic assistive system 200. For example, processor 206 may be a central processing unit (CPU) that may integrate with other hardware to provide processing capabilities for neuropathic assistive system 200. In another embodiment, processor 206 is a microcontroller. Processor 206 may be any device capable of processing functionality such as a system on chip (SoC), integrated circuit (IC), microcontroller (μC), application specific integrated circuit (ASIC), and the like, all may be operable to accept an input, execute a program, and return an output. Processor 206 may be pre-programmed to execute certain programs, or may be operable to be programmed by a user, and another using neuropathic assistive system 200, and a doctor/practitioner supervising a user's use of neuropathic assistive system 200. As used herein, “another” may indicate another user that may assist on a primary user's behalf, for example, a caretaker, home nurse, spouse, and the like. In one embodiment, processor 206 may be programmed to execute an instruction set stored on a computer readable medium, for example memory 208, that may cause sensor signals to be stored in memory 208 to await further processing by processor 206. Memory 208 may be integrated with processor 206 or be remote from processor 206. In another embodiment, processor 206 may be programmed to modulate an sensor signal from sensor 102, to communicate sensor signal to another wireless communication hardware. Processor 206 may be programmed to retrieve a sensor signal from sensor 206, process sensor signal to determine at least one of: an excess value, and a normal value, and, in response to an excess value, generate and transmit and actuator signal. Processor 206 may output signals as at least one of: a current, a voltage, an audio signal, and a video signal. Output from processor 206 may be in either an analog or digital format. Processor 206 may also be operable to demodulate communication signals and further process demodulated communication signals, so as to program control system 204 and processor 206, re-program control system 204 and processor 206, and adjust other parameters of components used in neuropathic assistive system 200. Processor 206 may accept inputs from other components, for example, through input 210, and provide outputs to other components, for example, through output 212. In one embodiment, processor 206 may comprise an integrated input 210, and output 212. In another embodiment, processor 206 may be operatively connected to a remote input 210, and remote output 212, for example, as a jack on case/housing 232 of neuropathic assistive system 200. Processor 206, memory 208, input 210, and output 212, may be compartmentalized as control system 204, so as to provide modular functionality of control system 204 in neuropathic assistive system 200—that is, control system may be easily arranged within neuropathic assistive system 200, swapped with another, removed, and duplicated.

Memory 208, as previously stated, may be used for storing sensor signals from sensors 202, and store an executed instruction set to be executed by processor 206, for example, to perform a method. Memory 208 may be integrated with processor 206, or may be remote from processor 206, and interconnected to processor 206, via connection 230. In one embodiment, memory 208 may be a volatile, random-access memory (RAM). In another embodiment, memory 208 may be a non-volatile read-only memory (ROM). In another embodiment, memory 208 may be a non-volatile random-access memory (NVRAM) such as a flash memory. Memory 208 may be physically attached to neuropathic assistive system 200, or memory 208 may be selectively removable from neuropathic assistive system 200, for example, such as a flash drive and flash memory card (i.e. secure digital (SD) or miniSD card).

Wireless communication hardware 214a may be integrated with control system 204, or wireless communication hardware 214a may be wholly separate from control system 204, as a stand-alone component, for example, as a separate IC interconnected to control system 204 via interconnection hardware 230. In one embodiment, wireless communication hardware 214a may comprise transmit/receive device 216a, and antenna 218b to form a modular component that may be easily moved, duplicated, swapped, and removed from neuropathic assistive system 200. Transmit/receive device may be at least one of: a transmitter, a receiver, and a transceiver. Antenna 218a may be used to wirelessly transmit RF energy signals, and wirelessly receive RF energy signals and move signals via interconnection hardware 230 to other components of neuropathic assistive system 200. Wireless communication hardware 214a may be operable to receive a communication signal, and provide a received communication signal to control system 204 as an input. Likewise, wireless communication hardware 214a may be used to transmit signals output from control system 204 as communication signals. As used herein, communication signals may be RF energy signals that may be transmitted wirelessly. Wireless communication hardware 214a may be able to modulate/demodulate control systems 204 outputs and received communication signals, or alternatively, modulation/demodulation may be performed by control system 204. Wireless communication hardware 214a may use any common communication standard such radio waves (of all allowable frequency ranges), Bluetooth®, Wi-Fi, and the like. Communication signals received by wireless communication hardware 214a may be used to control and program control system 204, and thus vary parameters and functionality of neuropathic assistive system 200.

Alert device 220 may be any device used to alert a user, a doctor/practitioner, and another, to a presence of at least one of: an inflammation condition, a wound, and a wound-causing environmental factor, otherwise recognizable by a user with a neuropathic condition due to diminished sensory capacity. Alert device 220 may be actuated by an output signal, for example, an actuator signal, sent from control system 204 that may comprise at least one of: a voltage, a current, an audio signal, and a video signal, in response to a processed sensor signal output from sensor 202. For example, sensor 202 may detect a wound-causing environmental factor and control system 204 may determine that a sensor signal of a detected wound-causing environmental condition may exceed a certain threshold value, which may cause control system 204 to output at least one of: a current, a voltage, an audio signal, and a video signal in response to an exceeded threshold value. In one embodiment, alert device 220 may comprise a display 222 to output a visual alert output. Visual alert output 222 may range from a simple electronic visual display system to a complex electronic visual display systems. In one embodiment, visual alert output 222 may be a colored light that may flash at a certain frequency to indicate that a sensor signal relating to at least one of: an inflammation condition, a wound, and a wound-causing environmental factor, may exceed a predetermined threshold value. In another embodiment, alert device 220 may be a remote device, for example, a smartphone, that may display a complex visual indication, for example, a foot map that may show both location and frequency of wound-causing environmental factors. In this embodiment, control system 206 may output a video signal that may be modulated and sent via wireless communication hardware 214a to a user's smart phone, for example, to alert a user to a presence of at least one of: an inflammation condition, a wound, and a wound-causing environmental factor. Alert device 220 may also comprise an audio alert output 226. Audio alert output 226 may be a simple speaker outputting at least one of: a noise, a tone, a buzz, programmed and/or recorded speech, and the like, in response to a sensor signal related to at least one of: an inflammation condition, a wound, and a wound-causing environmental factor. Alert device 220 may comprise a tactile alert output 224. Tactile alert output 224 may be an electro-mechanical device such as a motor or vibrator (i.e. vibration motor) which may provide a user with a tactile alert such as a vibration, buzz, tap, and the like, when a sensor signal relating to at least one of: an inflammation condition, a wound, and a wound-causing environmental factor may exceed a predetermined threshold value. Alert device 220 may be integrated in a case/housing 232 of neuropathic assistive system 200, or may be remote from neuropathic assistive system 200. For example, alert device 220 may be a tactile alert output 224 within a wearable item, such that a sensor signal exceed a predetermined threshold value may provide a tactile alert to a user through a clothing item with integrated neuropathic assistive system 200. In one embodiment, upon determination that a sensor signal may exceed a predetermined threshold value, a powerful tactile output is provided to a user, for example through a wearable insole, to alert a user with compromised sensory capacity of a presence of at least one of: an inflammation condition, a wound, and a wound-causing environmental factor. In another embodiment, alert device 220 may be a tactile alert device 224 shaped like watch and worn around a patient's wrist (not shown) that may provide a vibration to a patient's wrist when a presence of at least one of: an inflammation condition, a wound, and a wound-causing environmental may be detected by sensor 202. Other embodiments of alert device 220 may be contemplated such as a jewelry (i.e. pendant, ring), a headphone, a clothing item (i.e. belt, strap, hat), a fob, a smartphone, eyewear, and the like. Alert device 220 may be a modular component comprising at least one of: visual alert output 222, tactile alert output 224, and audio alert output 226. Alert device 220 may be easily added, removed, duplicated, and swapped as needed in neuropathic assistive system 200.

Power source 228a may be used to provide power to various components of neuropathic assistive system 200. Power source 228a may be a battery, rechargeable battery, capacitor, and the like to provide power to neuropathic assistive system 200. In one embodiment, power source 228a may be a generator that may use kinetic energy to generate power that may power neuropathic assistive system 200.

Interconnection hardware 230 may be any connection common for interconnection of electrical components. Interconnection hardware 230 may be at least one of: a wired connection, a wireless connection, and a combination of a wired and a wireless connection. In one embodiment, interconnection hardware 230 may be a bus system utilizing a common bus standard. In another embodiment, interconnection hardware 230 may be a wireless connection, for example, that may interconnect components with a Bluetooth® wireless transmission standard.

Neuropathic assistive system 200 may be packaged—that is, neuropathic assistive system 200 may comprise at least one of: a case/housing 232 and a support 234. In one embodiment, case/housing 232 may be substantially robust to limit an ingress of dust, water, and the like, to protect components of neuropathic assistive system 200 housed within case/housing 232. Support 234 may be a common support such as a printed circuit board that may provide an organized layout and support lines/vias 230 to interconnect components. In one embodiment, support 234 may be rigid. In another embodiment, support 234 may be flexible, allowing all components on support 234 to flex with support 234. In another embodiment, packaging of neuropathic assistive system 200 may not include case/housing 232, and may include support 234 only.

Neuropathic assistive system 200 may comprise attachment hardware 236 for easily attaching neuropathic assistive system 200 to at least one of: directly to a user; to a clothing item of a user; to an item in contact with a user; and to a user's medical device, for example, a wheelchair. Attachment hardware may comprise an elasticized band to fit around an arm, a leg, and a torso of a user, a hook and loop fastener to attach to a corresponding hook and loop fastener, a buckle that may be weaved onto a belt of a user, a clip, a pin, a snap, a button, and like hardware.

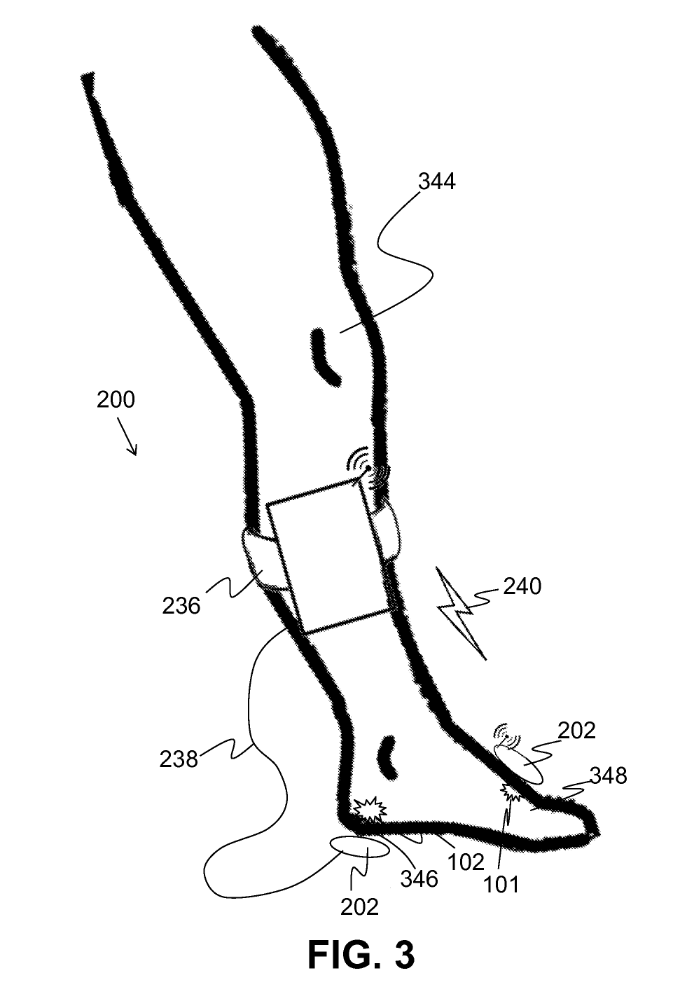

With reference to FIG. 3, a perspective view of an example neuropathic assistive system 200 is illustrated. In one embodiment, neuropathic assistive system 200 may be attached relatively near an affected tissue to sense, detect, and alert a user to a presence of at least one of: an inflammation condition 101, a wound 102, and a wound-causing environmental factor, such that location of neuropathic assistive system 200 may be selected to minimize a distance as may be used for wired connection 238. In another embodiment, neuropathic assistive system 200 may be attached relatively near an affected tissue, to minimize a distance of wireless connection 240. Neuropathic assistive system 200 may be operable to selectively attach, for example, to a surrounding area such as leg 344 via attachment hardware 236 to provide neuropathic assistive system 200 in close proximity to an affected tissue. Leg 344, for example, may not be affected by neuropathic conditions, such that a user's somatic senses and tactile perception may not be limited in tissue on leg 344. In one embodiment, neuropathic assistive system 200 may provide wired connection 238 of sensor 202 to a nearby affected tissue, for example wired sensor 202 attached directly to, or in proximity to inflammation condition/wound 102 on heel 346. In another embodiment, neuropathic assistive system 200 may provide wireless connection 240 of sensor 202 to a nearby affected tissue, for example wireless sensor 202 attached directly to, or in proximity to inflammation condition/wound 102 on bridge/superior surface of toes 348. In another embodiment, neuropathic assistive system 200 may provide wireless connection 240 of sensor 202 to a nearby affected tissue, for example wireless sensor 202 attached directly to, or in proximity to inflammation condition/wound 102 on the sole of one's foot.

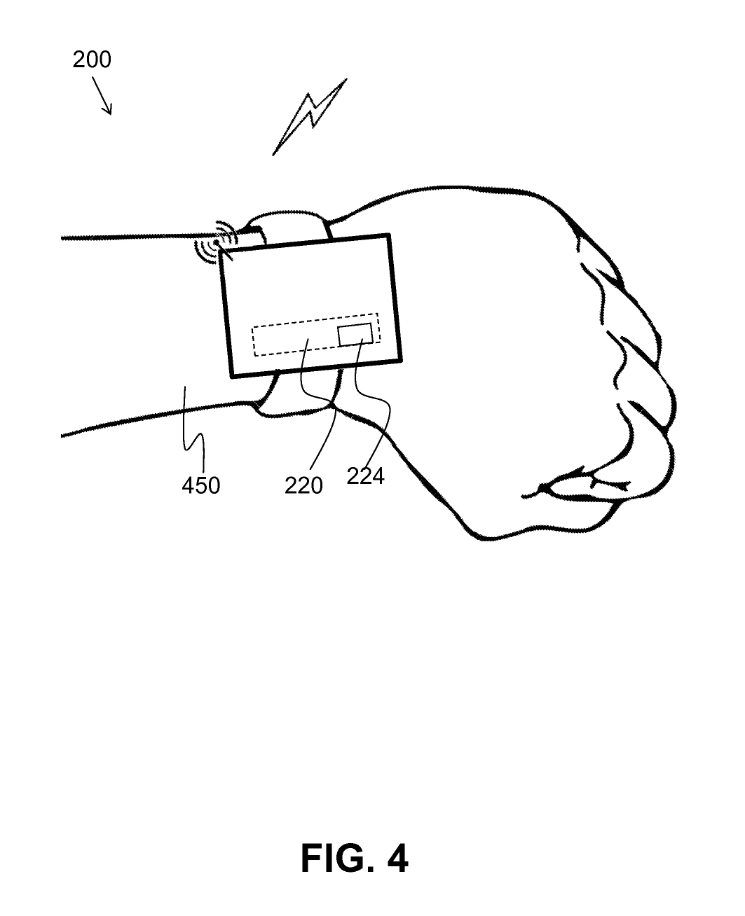

Referring now to FIG. 4, an example neuropathic assistive device 200 is illustrated. Neuropathic assistive device 200 may be at a location, for example, a user's wrist/arm 450 that may be further from an affected tissue (i.e. a user's foot), and operable to wirelessly receive sensor signals from a sensor. Neuropathic assistive device 200, or modular components thereof, such as alert device 220, may be at a location that is: more convenient to receive, for example, at least one of: a visual alert, a tactile alert, and an audio alert; and at a location of, or in contact with, tissue unaffected by neuropathic conditions such that a user may receive and sense a tactile alert from tactile alert output 224, for example, on wrist/arm 450.

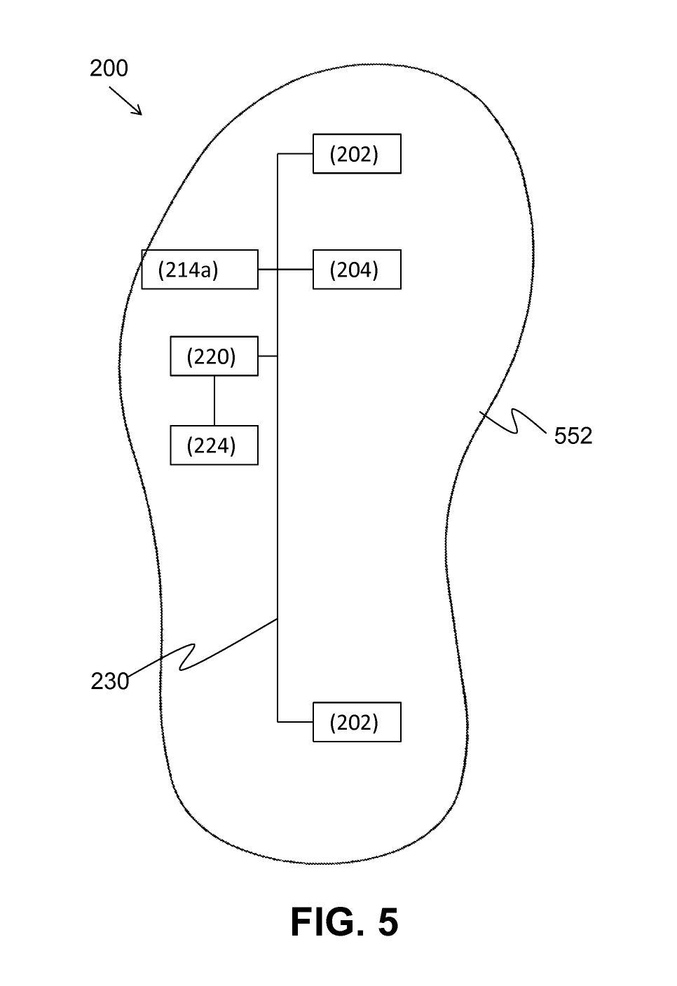

With reference to FIG. 5, an example neuropathic assistive system 200 may be integrated into a wearable item 552 or other item in close proximity to an affected tissue such as, for example, a removable insole. Various modular components may be included in neuropathic assistive system 200 depending on a user's needs. In one embodiment, neuropathic assistive system 200 embedded in wearable item 552 may comprise: wireless communication hardware 214a to receive a sensor signal from sensor 202, control system 204 to receive and process sensor signal from sensor 202 to determine an output; and alert device 220 that may output an alert in response to an output from control system 204, for example, a tactile alert from tactile alert output 224. In another embodiment, neuropathic assistive system 200 may comprise sensor 202 embedded in wearable item 552 such that a sensor may be in direct contact with, or in close proximity to, an affected tissue. Neuropathic assistive system 200 embedded in wearable item 552 may provide a user with an increased level of discretion such that a user may use neuropathic assistive system 200 without disturbing, or alerting others. Use of neuropathic assistive system 200 embedded in wearable item 552, may allow for an increased portability of neuropathic system 200 by allowing a user to transfer neuropathic assistive system 200 embedded in wearable item 552, for example, from one footwear to another, such that a user may use neuropathic assistive system 200 embedded in wearable item 552 with different pairs of shoes. In one embodiment, neuropathic assistive system 200 may be a custom fit orthotic 552 for a diabetic patient. In another embodiment, neuropathic assistive system 200 may be permanently integrated into a sole (outsole), a heel, and an upper (backstay, tongue, vamp, quarter, etc.) of a shoe. Neuropathic assistive system 200 embedded in wearable item 552 may be embedded in at least one of: a top, for example, a shirt; a bottom, for example, a pant and an undergarment; a headwear, for example, a hat and a headband; and a footwear, for example, a shoe, a sock, and an insole. In one embodiment, neuropathic assistive system 200 may be integrated into, and embedded in, wearable item 552, for example, between two layers of material. In another embodiment, neuropathic assistive system 200 may be operably connected to wearable item 552, for example, on an outer surface of wearable item 552.

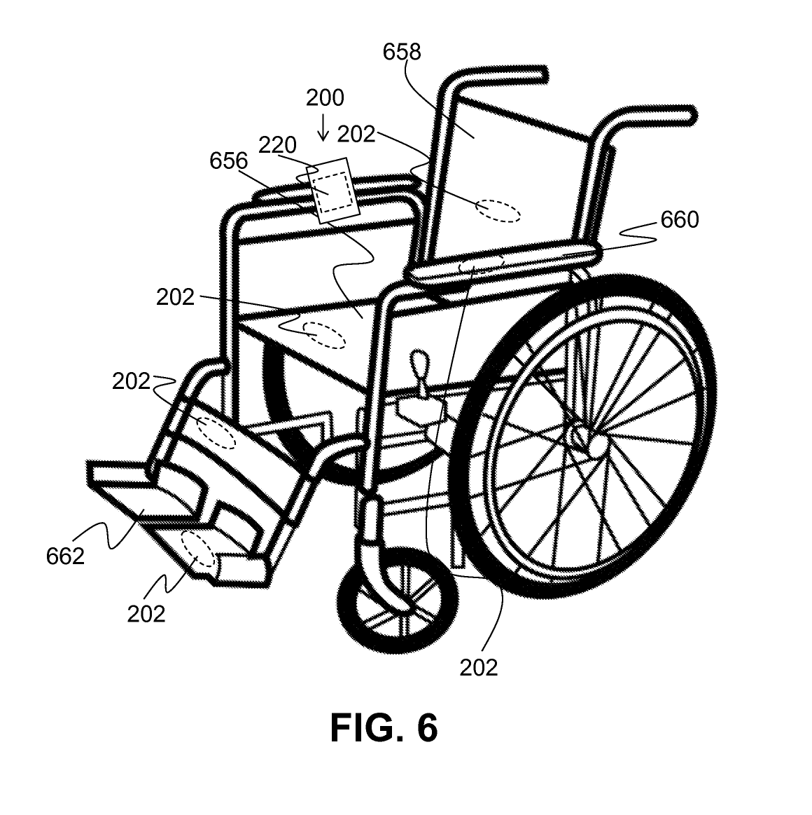

With reference to FIG. 6, a neuropathic assistive system 200 integrated into, or operably attached to a medical device 654 is illustrated. In one embodiment, medical device 654 may be a wheelchair 654. Disabled, bedridden, immobile, wheelchair-bound, and users with like conditions, may be particularly vulnerable to developing pressure-related ulcers. Integrating neuropathic assistive system into medical device 654 may better alert particularly vulnerable users to a presence of at least one of: an inflammation condition, a wound, and a wound-causing environmental factor. In one embodiment, sensor 202 may be integrated into, or on a surface of at least one of: seat 656, back 658, armrest 660, and footplate 662, on wheelchair 654. Sensor 202 may be positioned on medical device 654 so as to directly contact, or operationally contact (i.e. may be in close proximity to), an affected tissue. Sensor 202 may sense a location, a position, a displacement, and the like, relative to time such at an alert may be output from alert device 220 if an immobile user may not have moved an affected tissue within a threshold value of time. In one embodiment, certain modular components of neuropathic assistive system 200, for example, alert device 220 may be positioned in a location convenient to a user of medical device 654, for example, on armrest 660.

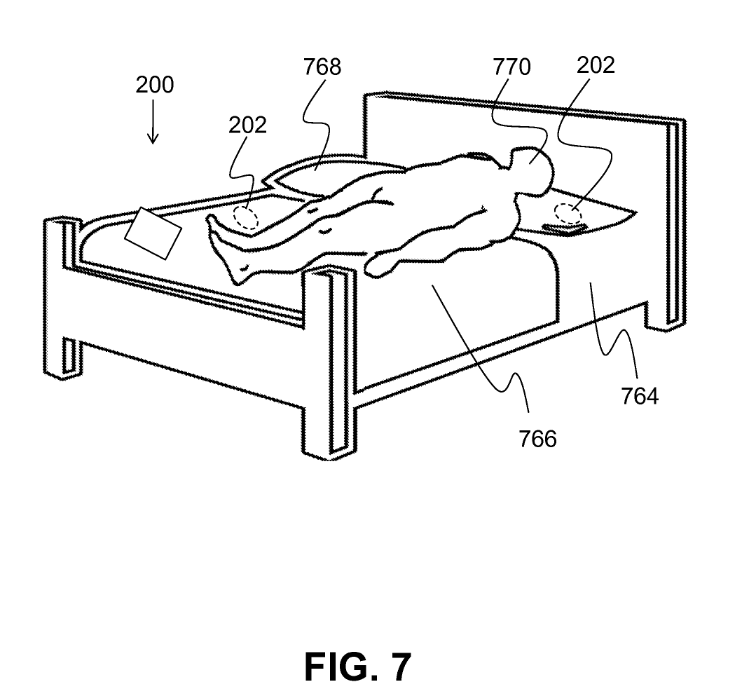

With reference to FIG. 7, neuropathic assistive system 200 may be integrated into, or operably attached to a bed linen or bed fixture. In one embodiment, a sensor 202 of neuropathic assistive system 200 may be integrated into a bed linen such as sheet 766, or pillowcase on pillow 768, such that sensor 202 may sense and detect a presence of at least one of: an inflammation condition, a wound, and a wound-causing environmental factor, for bedridden users. For example, sensor 202 of neuropathic assistive system 200 may be integrated into a pillowcase on pillow 768 to sense and detect a presence of at least one of: an inflammation condition, a wound, and a wound-causing environmental factor, on a neck/head 770 of a user. In another embodiment, sensor 202 may be embedded within a bed fixture such as mattress 764, and pillow 768, and may detect wound-causing environmental factors, such as an amount of pressure exerted by tissue of a user, relative to a bed fixture. For example, sensor 202 may be embedded in mattress 764 and may be used to measure an amount of pressure a tissue of a user may be exerting on mattress 764 to assess whether a sensed pressure level may cause an inflammation condition, or further exacerbate a wound. In one embodiment, sensor 202 may provide a location value relative to time that may cause an alert to be output from alert device 220 if a bedridden user may not have moved an affected tissue within a threshold value of time. In one embodiment, portions of neuropathic assistive system 200 may be embedded into a bed linen or bed fixture, for example, located between fabric layers of sheet 766. In another embodiment, portions of neuropathic assistive system 200 may be at a location on a surface of a bedlinen or a bed fixture, for example, on an outer surface of sheet 766 and may be in operable contact with a an affected tissue of a user.

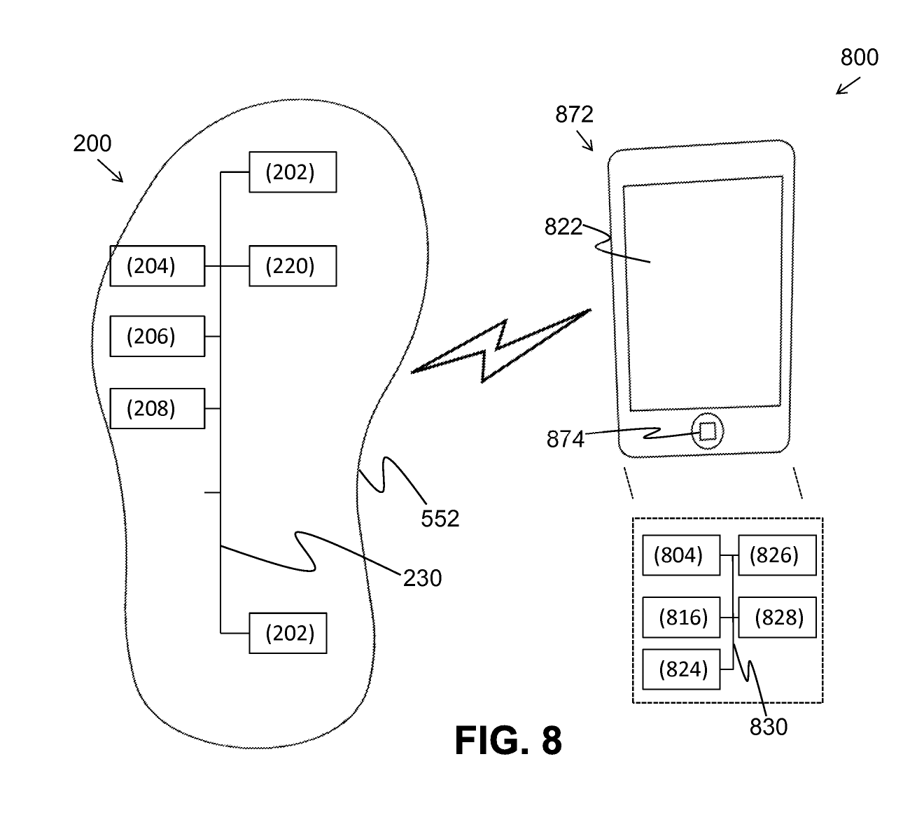

Referring now to FIG. 8, an example neuropathic assistive system 800 is illustrated. Neuropathic assistive system 800 may include a local neuropathic assistive system 200 and a remote neuropathic assistive system 872. In one embodiment, a local neuropathic assistive system 200, as described and illustrated in FIG. 1 and embedded in item 552 in FIG. 5, may interface with a remote neuropathic assistive device such as a smartphone 872, though any embodiment of local neuropathic assistive system 200 may interface with remote neuropathic assistive device 872. Remote device 872 may also be a remote, a fob, a PDA, a tablet computer, a smartwatch, a laptop computer, a personal computer, another portable electronic, and the like. Smartphone 872 may include display 822 for displaying an alert, a visual indication, and a graphical user interface (GUI) that may interface with local neuropathic assistive system 200. Smartphone 872 may include various input/output interfaces 874 such as buttons and a capacitive touch screen that may be used for inputting commands and parameters into GUI. Smartphone 872 may also include internal components such as, but not limited to a second control system 804 further comprising: a processor, a memory, an input, and an output; a second wireless hardware 816 further comprising an antenna, a tactile alert output 824, audio alert output 826, power source 828, interconnection hardware 830, and a jack. Second control system 804, second wireless hardware 816, display 822, tactile alert output 824, audio alert output 826, power source 828, interconnection hardware 830, a jack, and I/O interfaces 874, may be referred to collectively as “components” of remote neuropathic assistive device 872. Components of remote neuropathic assistive device 872 may share like, or similar functionality with like components of local neuropathic assistive device 200. As local neuropathic assistive device 200 may provide redundant functionality of components on remote neuropathic assistive device 872, for example, alert device 220, components in local assistive device 200 may be reduced based on system design, and needs of a user. Components for local neuropathic assistive system 200 may be reduced when used with remote neuropathic assistive device 872. For example, display 822, tactile alert output 824, and audio alert output 826 on remote neuropathic assistive device 872 may be used in place of alert device 220 in local neuropathic assistive system 200 to avoid duplicative and redundant use of components used in both local neuropathic assistive system 200 and remote neuropathic assistive device 872.

A GUI on remote neuropathic assistive device 872 may be a custom application (i.e. smartphone app) customized to a remote neuropathic assistive device 872 by operating system and device, for example an Android™ operating system on an Android-based device. Such a GUI may be used by a user, and a doctor/practitioner to interface with at least one of: sensor 202, control system 204, processor 206, and second control system 804, for controlling local neuropathic assistive system 200. For example, a GUI on remote neuropathic assistive device 872 may allow at least one of: a user, a doctor/practitioner, and another, to set a threshold value for sensor signals relative to a presence of at least one of: an inflammation condition, a wound, and a wound-causing environmental factor, as may be sensed and detected by sensor 202. GUI may also provide a visual alert, for example, on display 822 of remote neuropathic assistive device 872, to alert at least one of: a user, a doctor/practitioner, and another, to a presence of at least one of: an inflammation condition, a wound, and a wound-causing environmental factor, in place of an alert from alert device 220. Similar functionality of alert device 220—that may be: a visual alert, a tactile alert, and an audio alert, may be provided by display 822, vibrator motor 824, and speaker 826, all of which may be common components on remote neuropathic assistive device 872, for example, components commonly found on a smart phone 872. GUI may also be operable to display historical data such as sensor signals that may have been saved into at least one of: memory 208, and a memory from second control system 804. In this embodiment, GUI may display such indicators as: a frequency of detecting a presence of at least one of: an inflammation condition, a wound, and a wound-causing environmental factor; a location of at least one of: an inflammation condition, a wound, and a wound-causing environmental factor; a duration of tissue exposure to at least one of: an inflammation condition, a wound, and a wound-causing environmental factor; sensor signal values exceeding a predetermined threshold value in sensing and detecting at least one of: an inflammation condition, a wound, and a wound-causing environmental factor; and sensor signal values below a threshold value in sensing and detecting at least one of: an inflammation condition, a wound, and a wound-causing environmental factor. GUI may use other graphical indicators such as colors and symbols to show a historical trend of “positive actions” as generated by a user's actions to avoid and limit at least one of: inflammation conditions, wounds, and wound-causing environmental factors, as well as “negative actions” to alert at least one of: a user, a doctor/practitioner, and another, to actions that may have resulted in causing at least one of: an inflammation condition, a wound, as well as actions by a user that may expose a user to wound-causing environmental factors.

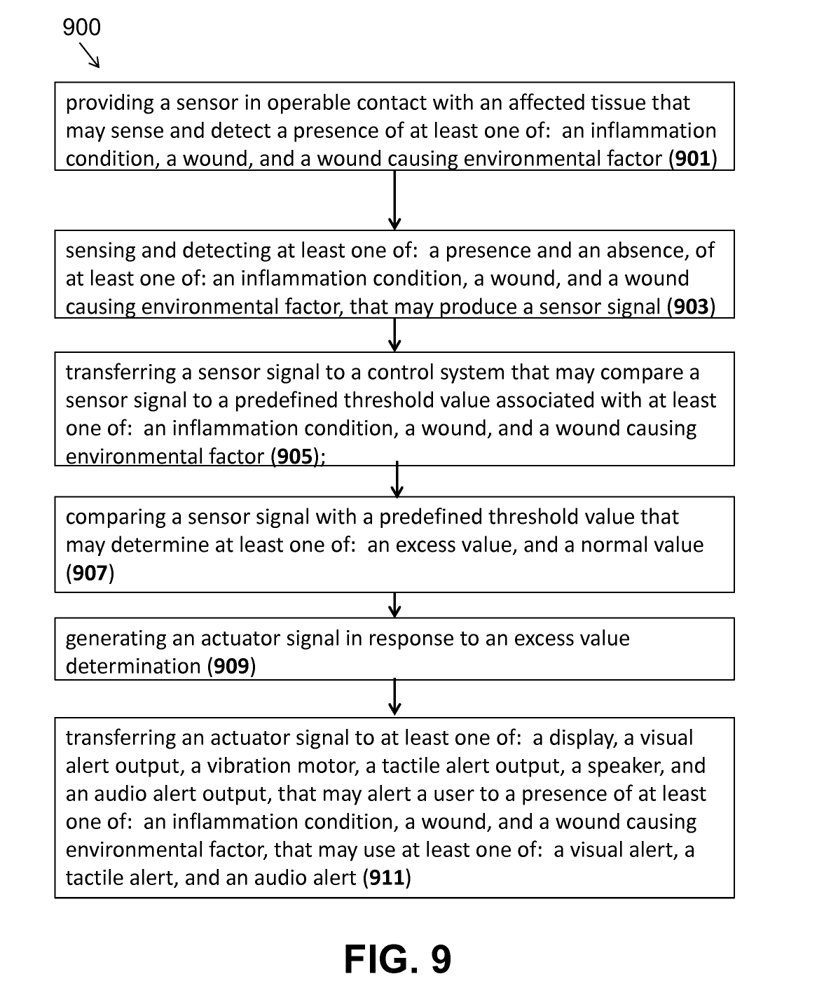

With reference to FIG. 9 a flowchart illustrating an example method of providing neuropathic assistance 900 is provided. Example method 900 may comprise: providing a sensor in operable contact with an affected tissue that may sense and detect a presence of at least one of: an inflammation condition, a wound, and a wound-causing environmental factor (901); sensing and detecting at least one of: a presence and an absence, of at least one of: an inflammation condition, a wound, and a wound-causing environmental factor, that may produce a sensor signal (903); transferring a sensor signal to a control system that may compare a sensor signal to a predefined threshold value associated with at least one of: an inflammation condition, a wound, and a wound-causing environmental factor (905); comparing a sensor signal with a predefined threshold value that may determine at least one of: an excess value, and a normal value (907); generating an actuator signal in response to an excess value determination (909); and transferring an actuator signal to at least one of: a display, a visual alert output, a vibration motor, a tactile alert output, a speaker, and an audio alert output, that may alert a user to a presence of at least one of: an inflammation condition, a wound, and a wound-causing environmental factor, that may use at least one of: a visual alert, a tactile alert, and an audio alert (911).

Unless specifically stated to the contrary, the numerical parameters set forth in the specification, including the attached claims, are approximations that may vary depending on the desired properties sought to be obtained according to the exemplary embodiments. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Furthermore, while the systems, methods, and apparatuses have been illustrated by describing example embodiments, and while the example embodiments have been described and illustrated in considerable detail, it is not the intention of the applicants to restrict, or in any way limit, the scope of the appended claims to such detail. It is, of course, not possible to describe every conceivable combination of components or methodologies for purposes of describing the systems, methods, and apparatuses. With the benefit of this application, additional advantages and modifications will readily appear to those skilled in the art. Therefore, the invention, in its broader aspects, is not limited to the specific details and illustrative example and exemplary embodiments shown and described. Accordingly, departures may be made from such details without departing from the spirit or scope of the general inventive concept. Thus, this application is intended to embrace alterations, modifications, and variations that fall within the scope of the appended claims. The preceding description is not meant to limit the scope of the invention. Rather, the scope of the invention is to be determined by the appended claims and their equivalents.

As used in the specification and the claims, the singular forms “a,” “an,” and “the” include the plural. To the extent that the term “includes” or “including” is employed in the detailed description or the claims, it is intended to be inclusive in a manner similar to the term “comprising,” as that term is interpreted when employed as a transitional word in a claim. Furthermore, to the extent that the term “or” is employed in the claims (e.g., A or B) it is intended to mean “A or B or both.” When the applicants intend to indicate “only A or B, but not both,” then the term “only A or B but not both” will be employed. Similarly, when the applicants intend to indicate “one and only one” of A, B, or C, the applicants will employ the phrase “one and only one.” Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. Finally, where the term “about” is used in conjunction with a number, it is intended to include ±10% of the number. In other words, “about 10” may mean from 9 to 11.

What is claimed:

1. A neuropathic assistance system comprising:
a sensor in operable contact with an affected tissue and operable to sense and detect a presence of at least one of: an inflammation condition, a wound, and a wound-causing environmental factor, at a location on or near the affected tissue;
a control system; and
an alert device.

2. The neuropathic assistance system of claim 1, wherein the sensor is at least one of: a capacitive-based sensor, a resistive-based sensor, a force sensor, a flex sensor, biomedical sensor; an EMG sensor, a wearable electrode, a magnetic sensor, a Hall-effect sensor, an RFID sensor, a temperature sensor, a moisture sensor, a tribo-sensor, a proximity sensor, a chemical sensor, a biological sensor, a pH level sensor, and a photodetector.

3. The neuropathic assistance system of claim 1, wherein the wound-causing environmental condition is at least one of: a force, a pressure, a contact, a friction, a flex, a moisture, a fluid, a movement, a temperature, a proximity, an electrodermal activity, a chemical presence, a biological presence, a pH level, and a color change.

4. The neuropathic assistance system of claim 1, wherein the alert device is at least one of: an actuator, a display, a visual alert output, a speaker, an audio alert output, a vibration motor, and a tactile alert output.

5. The neuropathic assistance system of claim 1, wherein control system further comprises at least one of: a processor, a memory, an input, and an output, wherein the control system is further operable to read and store a sensor signal from the sensor, and compare the sensor signal with a predetermined threshold value to determine at least one of: an excess value, and a normal value, and generating outputting an actuator signal in response to an excess value determination.

6. The neuropathic assistance system of claim 1, wherein the sensor, the control system, and the alert device are operatively connected to another by at least one of: a wired connection, and a wireless connection.

7. The neuropathic assistance system of claim 1, further comprising a wireless communication hardware.

8. The neuropathic assistance system of claim 1, further comprising a power source.

9. The neuropathic assistance system of claim 1, wherein the sensor further comprises an attachment hardware operable to attach the sensor to at least one of: a tissue on or near the affected tissue, and an item in operable contact with the affected tissue.

10. The neuropathic assistance system of claim 1, wherein the sensor further comprises: a wireless communication hardware, and a power source,
wherein the wireless communication hardware is operable to wirelessly transfer a sensor signal, and wherein the power source is operable to provide power to the sensor and the wireless communication hardware.

11. A neuropathic assistance system comprising:
a local neuropathic assistance system in operable contact with a user, the local neuropathic assistance system further comprising: a sensor in operable contact with an affected tissue and operable to sense and detect at least one of: an inflammation condition, a wound, and a wound-causing environmental factor, and provide a sensor signal; a first control system, an alert device, and a first communication hardware;
a remote neuropathic assistance system; the remote neuropathic assistance system operable to wirelessly receive the sensor signal, the remote neuropathic assistance system further comprising: a second control system, a second communication hardware, and at least one of: an electronic display, a vibration motor, and a speaker,
wherein the first communication hardware is operable to wirelessly communicate with the second communication hardware.

12. The neuropathic assistance system of claim 11, wherein the sensor is at least one of: a capacitive-based sensor, a resistive-based sensor, a force sensor, a flex sensor, biomedical sensor; an EMG sensor, a wearable electrode, a magnetic sensor, a Hall-effect sensor, an RFID sensor, a temperature sensor, a moisture sensor, a tribo-sensor, a proximity sensor, a chemical sensor, a biological sensor, a pH level sensor, and a photodetector.

13. The neuropathic assistance system of claim 11, wherein the wound-causing environmental condition is at least one of: a force, a pressure, a contact, a friction, a flex, a moisture, a fluid, a movement, a temperature, a proximity, an electrodermal activity, a chemical presence, a biological presence, a pH level, and a color change.

14. The neuropathic assistance system of claim 11, wherein the remote neuropathic assistance systems is one of: a smart phone, a tablet computer, a portable computing device, and a personal computer.

15. The neuropathic assistance system of claim 11, wherein each of the first control system and the second control system further comprises at least one of: a processor, a memory, an input, and an output.

16. The neuropathic assistance system of claim 15, wherein the remote neuropathic assistance system further comprises: an input/output interface; and a graphical user interface, the input/output interface operable to: provide a user input to control an operation of the graphical user interface, and control an execution of a computer readable instruction set to output a control signal from the second communication hardware, and wherein the second control system of the remote neuropathic assistance system may at least one of: store, and execute, at least one of: the computer readable instruction set, and the sensor signal.

17. The neuropathic assistance system of claim 11, wherein each of the sensor, the first control system, the first communication hardware, the alert device, the second control system, the second communication hardware, and the at least one of: the electronic display, the vibration motor, and the speaker, are operatively connected to each other by at least one of: a wired connection, and a wireless connection.

18. The neuropathic assistance system of claim 11, wherein each of the first communication hardware, and the second communication hardware further comprises at least one of: a transmitter, a receiver, a transceiver, and an antenna.

19. The neuropathic assistance system of claim 11, wherein each of the local neuropathic assistance system and remote neuropathic assistance system further comprises a power source.

20. A method for providing neuropathic assistance, the method comprising:
providing a sensor in operable contact with an affected tissue to sense and detect a presence of at least one of: an inflammation condition, a wound, and a wound-causing environmental factor;
sensing and detecting at least one of: the presence, and an absence, of at least one of: the inflammation condition, the wound, and the wound-causing environmental factor, to produce a sensor signal;
transferring the sensor signal to a processing device for comparison against a predefined threshold value associated with the at least one of: the inflammation condition, the wound, and the wound-causing environmental factor;
comparing the sensor signal against the predefined threshold value to determine at least one of: an excess value, and a normal value;
generating an actuator signal in response to an excess value determination; and
transferring the actuator signal to at least one of: a display, a visual alert out, a vibration motor, a tactile alert output, a speak, and an audio alert output, to alert a user to the presence of at least one of: an inflammation condition, a wound, and a wound-causing environmental factor, using at least one of: a visual alert, a tactile alert, and an audio alert.
